(19) United States
(12) Patent Application Publication
(10) Pub. No.: US 2009/0011005 A1
(43) Pub. Date: Jan. 8, 2009

(54) Pharmaceuticals compositions containing nanomaterials useful for treating restenotic lesions

(76) Inventors: Alexandre Do Canto Zago, Porto Alegre (BR); Alcides Jose Zago, Porto Alegre (BR)

Correspondence Address: PATENT LAW AGENCY, LLC, 3146 NORTH VERDUGO ROAD, GLENDALE, CA, 91208-1665, US

(21) Appl. No.: 12/217,028

(22) Filed: Jul. 1, 2008

Related U.S. Application Data
(63) Continuation of application No. PCT/BR07/00015, filed on Dec. 12, 2007.

(30) Foreign Application Priority Data
Mar. 8, 2007 (BR) PI 0600285-4

(51) Int. Cl. A61K 9/127; A61K 9/16; A61K 31/436; A61K 31/337

(52) U.S. Cl. 424/450; 424/489; 424/490; 514/291; 514/449; 977/773

(57) ABSTRACT

Pharmaceutical compositions containing nanomaterials useful for treating restenotic lesions, which comprise nanomaterials containing one or more antiproliferative agents for treatment of intra-stent restenotic lesions by means of a local infusion, providing the increase in adhesion, penetration, and diffusion of the nanomaterials that contain the antiproliferative active agent in the tissue responsible for the neointimal hyperplasia, are described.

### CROSS-REFERENCE TO RELATED APPLICATIONS

This Application is a continuation of a co-pending International Patent Application No. PCT/BR2007/000015 with an international filing date of 12 Dec. 2007 that designated the United States of America, which claims the benefit of priority of the Federative Republic of Brazil Patent Application No. PI 0600285-4, with a filing date of 13 Jan. 2006, the entire disclosures of all Applications is expressly incorporated by reference in their entirety herein.

### BACKGROUND OF THE INVENTION

1. Field of the Invention

This invention concerns pharmaceuticals containing nanomaterials for treatment of restenotic lesions. More specifically, it comprehends pharmaceuticals compositions that comprise nanomaterials containing one or more antiproliferative agents for treatment of intra-stent restenotic lesions by means of a local infusion.

2. Description of Related Art

The development of restenosis may be observed angiographically as a reduction in the coronary luminal diameter that occurs after the dilation of an obstruction.

Metal tubular structures known as stents are implanted in order to prevent the vessel from closing again. Although this technique significantly lessens the restenosis problem, it still persists. The blood flow ends up impaired due to the stent post-implant re-obstruction of the coronary artery that occurs because of the disordered and excessive growth of both endothelial and smooth muscle cells inside the stent.

Thus, the restenosis occurs in approximately 25% of the uncoated stent implant cases, a rate which may amount to 50% in accordance with a patient's clinical and angiographic characteristics of the obstructive lesion and of the coronary artery to be treated.

Recent studies point out that the restenosis rate may be significantly reduced by implanting stents coated with drugs capable of inhibiting the neontimal proliferation for a few weeks. Although these stents reduce the restenosis up to 8%, which is the smallest rate already achieved by a therapeutic device in coronary arteries, the restenosis persists and constitutes an important problem of difficult solution. Besides, the high cost of the drug-eluting stents limits their routine use in most of the countries.

Several techniques have been employed for the intra-stent restenosis treatment such as balloon-catheter angioplasty, cutting-balloon, catheter directed atherectomy, and laser. All of these techniques are very costly, highly complex, and do not determine results superior to those of the balloon catheter, which constitutes a more simple and less costly option.

Brachytherapy with gamma and beta radiation has also been studied as a technique for treating restenotic lesions. The initial results were very encouraging; however, the loss of the initial result was verified over time, which gives this technique a palliative effect. Other negative aspects of this technique are a very high cost and logistics because there is a need for a brachytherapy specialist during the performance of the procedure and radioactive sources of short duration in addition to protection shields and isolation of an area in case the gamma radiation is used. Therefore, brachytherapy is a technique which is practically no longer in use nowadays.

The use of stents coated with antiproliferative drugs is at present the best therapeutic strategy for treating restenotic lesions with the recurrence rate placed between 14 and 22%, however the high cost and not so satisfactory results such as those obtained when using these stents in the treatment of these de novo lesions, that is, treated virgin lesions, limit the wide-spread use of this therapeutic strategy.

The oral administration of rapamycin was also studied and showed the restenosis rate of approximately 22% when using high doses. The costs are reasonable although the results are not very satisfactory.

Rapamycin or sirolimus is a powerful antiproliferative agent that acts in the G1-S phase of the cell cycle. As an antiproliferative agent, it has also been used in coronary stents providing the significant reduction in the rates of intra-stent neointimal hyperproliferation called restenosis. This cell antiproliferative effect has been shown in several in vitro studies of animals and humans.

The application for the invention patent US20050244503 describes a pharmaceutical formulation containing nanoparticles of an anticonvulsive agent, which are coated with a surfactant such as a cationic one.

The application for the invention patent WO2006102378 describes a continuous method of delivering an active agent for treatment of the angiogenesis, the said agent including, but not limited to, rapamycin.

The technical literature presents products and methods that, in spite of reducing the new intra-stent restenosis rates, do not produce medium to long-term results that could be considered satisfactory. Therefore, there is still a need for development of pharmaceutical compositions that comprise nanomaterials containing one or more antiproliferative agents for treatment of intra-stent restenotic lesions by means of a local infuslion.

Thus, the public domain literature neither describes nor suggests pharmaceutical compositions that comprise nanomaterials containing one or more antiproliferative agents chosen from rapamycin or the like, and/or paclitaxel or the like, the said compositions being useful for treating intra-stent restenotic lesions by means of a local infusion; such compositions are described and claimed for in this application.

### BRIEF SUMMARY OF THE INVENTION

In general, this invention concerns pharmaceutical compositions that comprise nanomaterials containing one or more antiproliferative agents for treatment of intra-stent restenotic lesions by means of a local infusion.

The characteristic of the invention is pharmaceutical compositions that comprise nanomaterials containing one or more antiproliferative agents for treatment of intra-stent restenotic lesions by means of a local infusion.

The characteristic of the invention is pharmaceutical compositions that comprise nanomaterials containing one or more antiproliferative active agents chosen from rapamycin or the like, and/or paclitaxel or the like.

The characteristic of the invention is pharmaceutical compositions that comprise cation, anion or neutrally coated nanomaterials.

These and other features, aspects, and advantages of the invention will be apparent to those skilled in the art from the following detailed description of preferred non-limiting exemplary embodiments, taken together with the drawings and the claims that follow.

### BRIEF DESCRIPTION OF THE DRAWINGS

None.

### DETAILED DESCRIPTION OF THE INVENTION

The detailed description set forth below in connection with the appended drawings is intended as a description of presently preferred embodiments of the invention and is not intended to represent the only forms in which the present invention may be constructed and or utilized.

The pharmaceutical compositions containing nanomaterials useful for treating restenotic lesions, subject matter of this invention, comprise nanomaterials containing one or more antiproliferative agents for treatment of intra-stent restenotic lesions by means of a local infusion, providing the increase in adhesion, penetration, and diffusion of the nanomaterials that contain the antiproliferative active agent in the tissue responsible for the neointimal hyperplasia.

In the first modality, the pharmaceutical compositions comprise nanomaterials containing rapamycin (sirolimus) or the like, and nanomaterials containing paclitaxel or the like.

In the second modality, the pharmaceutical compositions comprise nanomaterials containing rapamycin (sirolimus) or the like, and paclitaxel or the like.

In the third modality, the pharmaceutical compositions comprise nanomaterials containing rapamycin (sirolimus) or the like.

In the fourth modality, the pharmaceutical compositions comprise nanomaterials containing paclitaxel or the like.

The nanomaterials are selected from among nanoparticles, nanocapsules, liposomes, nanotubes, nanospheres, or the like.

Preferably, the pharamaceutical compositions containing nanomaterials useful for treatment of restenotic lesions comprise anionic, neutral, or cationic nanoparticles. In all of the modalities of the invention, the nanomaterials may be cation, anion, or neutrallly coated.

The analogues of rapamycin (sirolimus) are chosen from among biolimus, everolimus, tacrolimus, zotarolimus, pimecrolimus or ascomycin.

The analogues of paclitaxel comprehend docetaxel.

The solution of nanomaterials containing an antiproliferative active agent chosen from among rapamycin or the like, and/or paclitaxel or the like, is infused at a concentration of from 0.001 mg of active agent/ml to 10 mg of active agent/ml due to the wide therapeutic range.

The method consists of the infusion of the pharmaceutical compositions containing nanomaterials useful for treating restenotic lesions, subject matter of this invention, in the wall of the coronary artery by means of a catheter specific for a local infusion of the drug; such procedure must be carried out after the stent has been dilated with a conventional balloon-catheter or a cutting-balloon.

The local infusion of nanomaterials containing one or more antiproliferative agents constitutes a therapeutic strategy technically of simple execution, potentially effective, and economically feasible for treatment of intra-stent restenotic lesions. In order to assess the results of these compounds in the treatment of restenotic lesions, a study was carried out on swine as described below:

The two solutions of nanoparticles were prepared, the first solution having nanoparticles containing rapamycin and with a cationic coating, and the second solution having nanoparticles containing rapamycin and without a cationic coating.

Twelve commercially available 3.0×16.0 mm stents were implanted at high pressure in the left anterior descending coronary artery of 2.75 mm in diameter of six swine considering that two stents were implanted per coronary artery—one in the transition of the proximal third to the medium one, and the other in the medium third. 30 days later, all of the swine were studied by cineangiocoronariography and intra-coronary ultrasound that revealed restenosis (obstruction superior to 50%) in all of the previously implanted stents. Then, the angioplasty with a conventional 3.0×16.0 mm balloon-catheter was performed in all of the stents, followed by the local infusion of nanoparticles containing rapamycin without cationic coating with a drug-infusion catheter in four stents, and of nanoparticles containing rapamycin with cationic coating in the other four.

60 days later, all of the swine were studied again by cineangiocoronariography and intra-coronary ultrasound that showed a medium-sized stenosis area of 63% in the stents treated only with conventional angioplasty, 20% in the stents treated with nanoparticles containing rapamycin without cationic coating, and 18% in the stents treated with nanoparticles containing rapamycin with cationic coating.

The obtained results evidenced a satisfactory effect of the local infusion of nanoparticles containing rapamycin with or without cationic coating in the prevention of recurrent episodes of restenosis after the treatment of the intra-stent restenosis. There is no significant difference in the use of nanoparticles containing rapamycin with cationic coating with relation to the nanoparticles containing rapamycin without cationic coating; however, a small advantage in favor of the nanoparticles with cationic coating was verified.

Although the invention has been described in considerable detail in language specific to structural features and or method acts, it is to be understood that the invention defined in the appended claims is not necessarily limited to the specific features or acts described. Rather, the specific features and acts are disclosed as preferred forms of implementing the claimed invention. Stated otherwise, it is to be understood that the phraseology and terminology employed herein, as well as the abstract, are for the purpose of description and should not be regarded as limiting.

It should further be noted that throughout the entire disclosure, the labels such as left, right, front, back, top, bottom, forward, reverse, clockwise, counter clockwise, up, down, or other similar terms such as upper, lower, aft, fore, vertical, horizontal, proximal, distal, parallel, perpendicular, etc. have been used for convenience purposes only and are not intended to imply any particular fixed direction or orientation. Instead, they are used to reflect relative locations and/or directions/orientations between various portions of an object.

In addition, reference to “first,” “second,” “third,” and etc. members throughout the disclosure (and in particular, claims) is not used to show a serial or numerical limitation but instead is used to distinguish or identify the various members of the group.

In addition, any element in a claim that does not explicitly state “means for” performing a specified function, or “step for” performing a specific function, is not to be interpreted as a “means” or “step” clause as specified in 35 U.S.C. Section 112, Paragraph 6. In particular, the use of “step of,” “act of,” “operation of,” or “operational act of” in the claims herein is not intended to invoke the provisions of 35 U.S.C. 112, Paragraph 6.

What is claimed is:

1. Pharmaceutical compositions containing nanomaterials useful for treating restenotic lesions characterized by nanomaterials containing rapamycin (sirolimus) or the like, and the nanomaterials containing paclitaxel or the like.

2. Pharmaceutical compositions containing nanomaterials useful for treating restenotic lesions characterized by nanomaterials containing rapamycin (sirolimus) or the like, and paclitaxel or the like.

3. Pharmaceutical compositions containing nanomaterials useful for treating restenotic lesions characterized by nanomaterials containing rapamycin (sirolimus) or the like.

4. Pharmaceutical compositions containing nanomaterials useful for treating restenotic lesions characterized by nanomaterials containing paclitaxel or the like.

5. Pharmaceutical compositions containing nanomaterials useful for treating restenotic lesions in accordance with the claims 1 through 4 characterized by the fact that the analogues of rapamycin (sirolimus) are selected from among biolimus, everolimus, tacrolimus, zotarolimus, pimecrolimus or ascomycin.

6. Pharmaceutical compositions containing nanomaterials useful for treating restenotic lesions in accordance with the claims 1, 2, and 3, characterized by the fact that the analogues of paclitaxel are docetaxel.

7. Pharmaceutical compositions containing nanomaterials useful for treating restenotic lesions in accordance with the claims 1 through 4, characterized by the fact that the nanomaterials are selected from among nanoparticles, nanocapsules, liposomes, nanotubes, nanospheres, or the like.

8. Pharmaceutical compositions containing nanomaterials useful for treating restenotic lesions in accordance with the claims 1 through 4, characterized by the fact that the nanomaterials are anionic, neutral or cationic.

9. Pharmaceutical compositions containing nanomaterials useful for treating restenotic lesions in accordance with the claims 1 through 4, characterized by the fact that the nanomaterials have a cationic, anionic, or neutral coating.
